(19) United States
(12) Patent Application Publication
(10) Pub. No.: US 2013/0066373 A1
(43) Pub. Date: Mar. 14, 2013

(54) BODY HAULING MECHANISM

(71) Applicant: Chung-Fong LIAO, New Taipei City (TW)

(72) Inventor: Chung-Fong LIAO, New Taipei City (TW)

(21) Appl. No.: 13/232,446

(22) Filed: Sep. 14, 2011

(51) Int. Cl. A61F 5/00

(52) U.S. Cl. 606/241

(57) ABSTRACT

A body hauling mechanism includes a weight seat, a weight control assembly and a body hauling auxiliary part. The weight control assembly of the body hauling mechanism has an enclosure with a stretching mechanism configured inside. Said stretching mechanism is made up of a separating board, a gear unit, a cord rolling wheel, a back-stop mechanism and a driving wheel. The user can pull the two-way pulling cord to drive the stretching mechanism to control the weight control assembly hanged on the weight seat, so that the hauling cord of the weight control assembly can be retracted or released mildly, and can apply or release the hauling force smoothly. When using the body hauling mechanism to improve the symptoms caused by irritation or compression of the body nerves, secondary injury of the affected part can be avoided.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates generally to a stretching mechanism, and more particularly to an innovative one which can improve the symptoms caused by stimulation or compression of cervical-shoulder nerves.

2. Description of Related Art Including Information Disclosed Under 37 CFR 1.97 and 37 CFR 1.98

Cervical-shoulder Syndrome is a generic term for various symptoms caused by stimulation or compression of cervical-shoulder nerves. Clinical symptoms include soreness or pain in the neck, spasm of the neck or shoulder muscles, stiffness of the neck and limitation of movement, or even the pain is transmitted to the shoulder, suprascapula, front arm and fingers, and feeling of inertia of the upper extremity and anesthesia of the fingers. Cervical-shoulder Syndrome happening to young people is mainly caused by improper posture, which, over a long period of time, imposes improper pressure on the intervertebra discs of the cervical vertebrae, and consequently, causes the intervertebra discs to slip back and impose pressure on and stimulate the hard film of the spinal cord and nerve roots. Cervical-shoulder Syndrome happening to middle and old aged people is mainly caused by nerve compression resulting from degeneration of the cervical vertebrae or hyperosteogeny.

To overcome the above problems, generally a cervical vertebrae hauling mechanism is used to improve the symptoms caused by Cervical-shoulder Syndrome. Said cervical vertebrae hauling mechanism has a cervical vertebrae hauling strap and a stretching and stopping unit. The cervical vertebrae hauling strap is connected to the lower end of the stretching and stopping unit and the strap-shaped part is looped onto the mentum of the user. The stretching and stopping unit comprises a pulley unit, a pulling cord and a releasing mechanism. The pulling cord is winded on the pulley unit. The user can pull downward the pulling cord to control the pulley unit to rise and drive the cervical vertebrae hauling strap to impose a preset stretching force on the affected part of the user. To release the stretching force on the affected part by the stretching and stopping unit, the user can pull downward the releasing cord of the releasing mechanism;

However, such a structural design has a problem that, when the releasing mechanism suddenly releases the stretching force, the affected part may suffer from secondary injury due to the sudden change of load. Hence, there is still room for improvement. The present invention aims to further improve the design through innovations.

Thus, to overcome the aforementioned problems of the prior art, it would be an advancement if the art to provide an improved structure that can significantly improve the efficacy.

Therefore, the inventor has provided the present invention of practicability after deliberate design and evaluation based on years of experience in the production, development and design of related products.

### BRIEF SUMMARY OF THE INVENTION

The facts of efficacy advancement of the present invention are:

The “body hauling mechanism” disclosed in the present invention adopts an innovative and unique structural design of a two-way pulling cord which can be pulled to drive the stretching mechanism to generate a hauling force or release the hauling force. Compared to the known structure of the prior art, the present invention offers a weight control assembly hanged on the weight seat. The stretching mechanism of the weight control assembly is made up of a gear unit, a cord rolling wheel, a back-stop mechanism and a driving wheel. When the user pulls the two-way pulling cord, the hauling cord of the weight control assembly can generate a hauling force or release the hauling force at a mild speed. When using the body hauling mechanism to improve the symptoms caused by irritation or compression of body nerves, the mild hauling speed can avoid secondary injury of the affected part.

Although the invention has been explained in relation to its preferred embodiment, it is to be understood that many other possible modifications and variations can be made without departing from the spirit and scope of the invention as hereinafter claimed.

### BRIEF DESCRIPTION OF THE SEVERAL VIEWS OF THE DRAWINGS

FIG. 1 is an assembled perspective view of the body hauling mechanism of the present invention.

FIG. 2 is an assembled perspective view of the weight control assembly of the present invention.

FIG. 3 is an exploded perspective view of the weight control assembly of the present invention.

FIG. 4 is a structural view of the present invention showing configuration of the gear unit, cord rolling wheel and two-way pulling cord of the stretching mechanism.

FIG. 5 is a structural sectional view of the weight seat of the stretching mechanism disclosed in the present invention.

FIG. 6 is a structural sectional view of the cord rolling wheel and hauling cord of the stretching mechanism disclosed in the present invention.

FIG. 7 is a structural view of the back-stop mechanism of the stretching mechanism disclosed in the present invention.

FIG. 8 shows a schematic view of an embodiment example of the method to use the body hauling mechanism of the present invention to improve cervical-shoulder symptoms.

FIG. 9 is a schematic view showing the working method to use the body hauling mechanism of the present invention to improve cervical-shoulder symptoms.

### DETAILED DESCRIPTION OF THE INVENTION

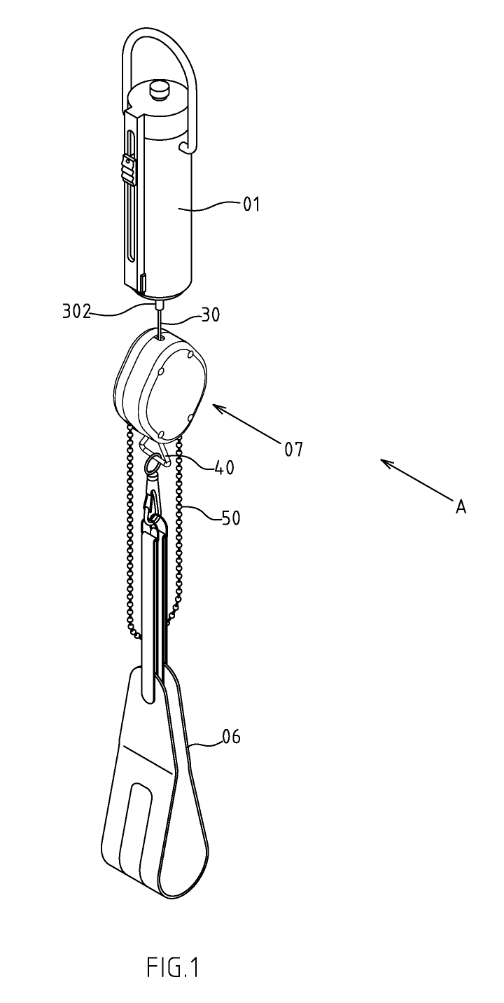

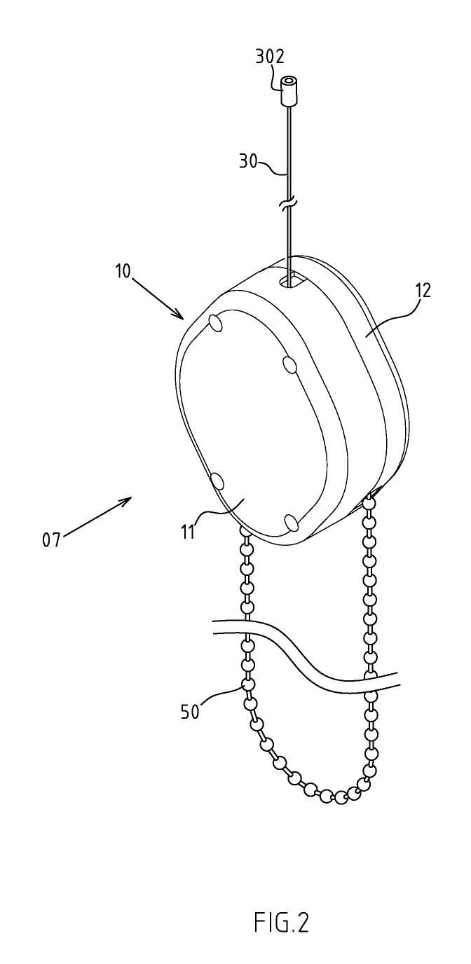

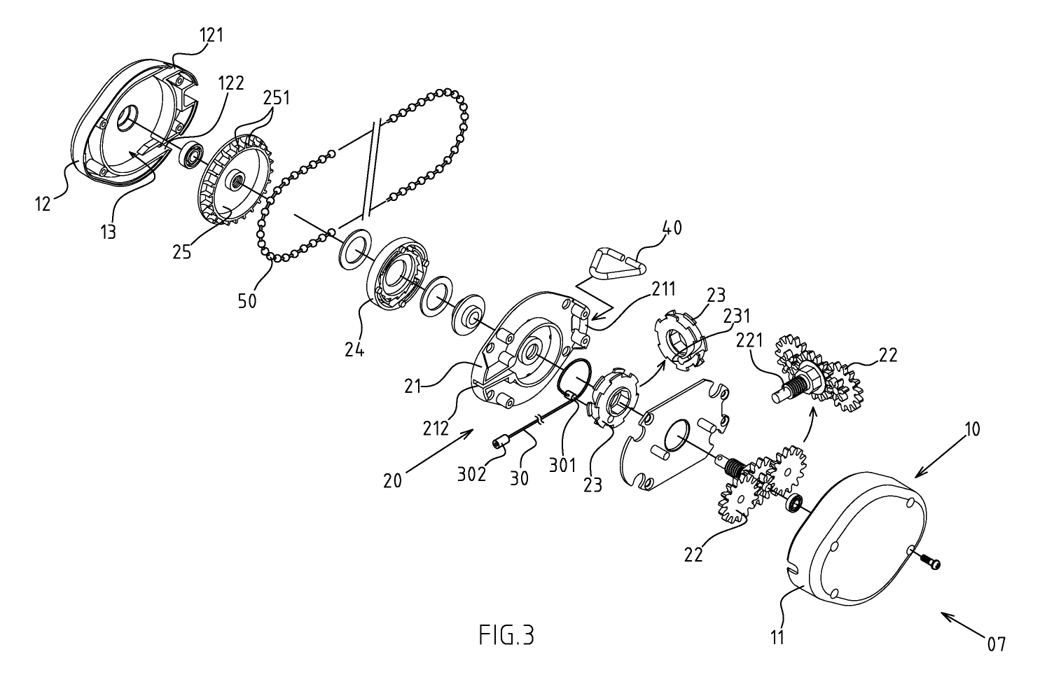

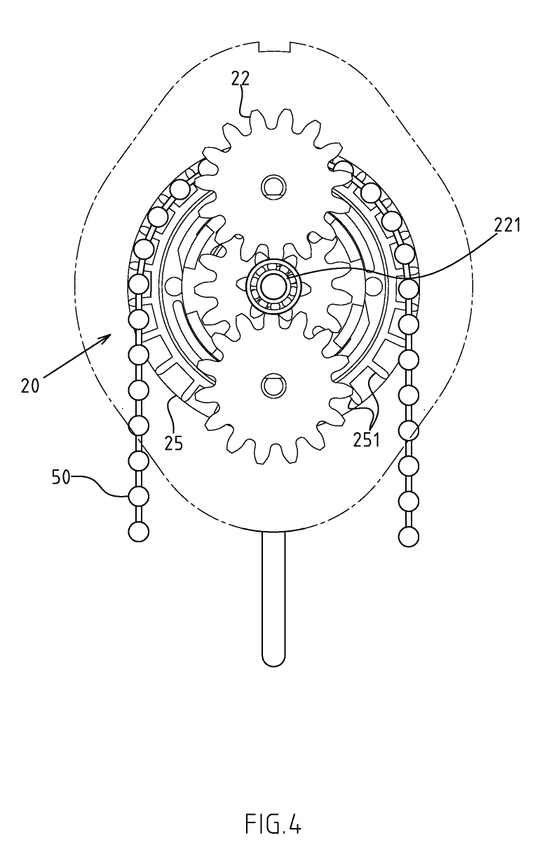

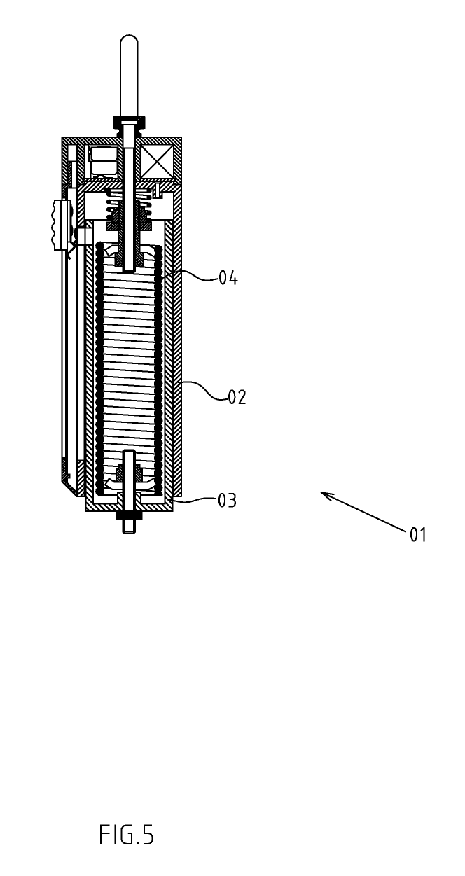

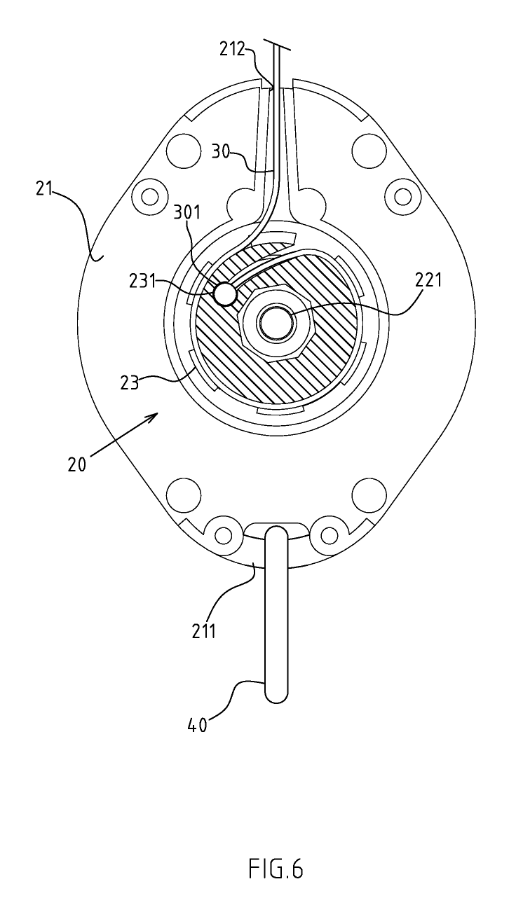

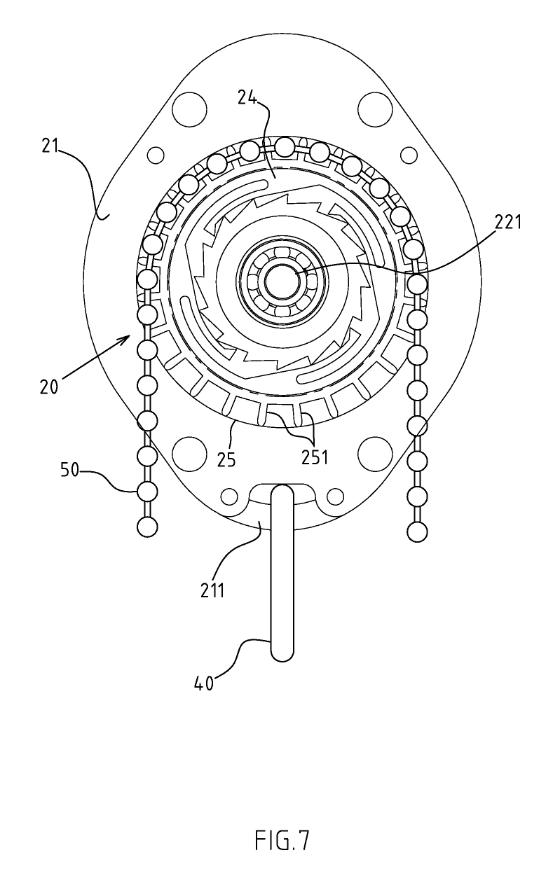

FIGS. 1-7 show the preferred embodiments of the body hauling mechanism of the present invention. However, such an embodiment is for description purpose only and the patent application shall not be limited to such a structure.

Said body hauling mechanism A comprises a weight seat 01, formed outside with an outer seat 02, and inside fitted with an inner seat 03 which can slip out of the bottom end of the outer seat 02, and, between the inner seat 03 and outer seat 02, a spring weight mechanism 04 is configured

A weight control assembly 07 includes an enclosure 10, comprising a front enclosure 11 and a rear enclosure 12 corresponding to each other and fixed together, and inside the front enclosure 11 and rear enclosure 12, a housing space 13 is formed. On one side of the rear enclosure 12, there is a first guiding hole 121 and a second guiding hole 122.

A stretching mechanism 20 is configured inside the housing space 13 of the enclosure 10.

A hauling cord 30 is configured on a preset position of the stretching mechanism 20, and formed on the end with a connecting part 302. The connecting part 302 is connected and fixed to the bottom of the inner seat 03 of the weight seat 01. Through activation of the stretching mechanism 20, the hauling cord 30 can be winded up or released.

A hanging unit 40 is configured on a preset position of the stretching mechanism 20 for the weight control assembly 07 to hang a body hauling auxiliary part 06.

A two-way pulling cord 50 is configured on a preset position of the stretching mechanism 20, which is a flexible cord and can be pulled in both directions. By pulling the two-way pulling cord 50, the stretching mechanism can be driven to wind up or release the hauling cord 30, so that the weight control assembly 07 can generate a hauling force forward or backward.

The two-way pulling cord 50 is structured in a chain of beads with uniform spacing, with its front end and terminal end connected together to form a loop.

The stretching mechanism 20 of the weight control assembly 07 comprises a separating board 21, a gear unit 22, a cord rolling wheel 23, a back-stop mechanism 24 and a driving wheel 25. The separating board 21 is fitted on the part where the front and rear enclosure 11, 12 are assembled together. On one side of the enclosure, a connecting part 211 is configured. On the side of the connecting part 211, a cord-guiding hole 212 is configured. The gear unit 22 is configured in front of the separating board 21, and on the rear end of the gear unit 22 a driving axis 221 is configured. Inside the space formed by the separating board 21 and the gear unit 22, a cord rolling wheel 23 is configured. The back-stop mechanism 24 is configured and fixed on the back side of the separating board 21 and can rotate in one direction, so as to avoid downside slipping of the weight control assembly 07 when the hauling cord 30 is loaded. On the back side of the back-stop mechanism 24, a driving wheel 25 is configured and can rotate in both directions. The driving axis 221 is serially connected to the cord rolling wheel 23, back-stop mechanism 24 and driving wheel 25, so that the cord rolling wheel 23, back-stop mechanism 24 and driving wheel 25 can be driven to rotate simultaneously, and the stretching mechanism 20 is entirely positioned inside the housing space 13 of the enclosure 10.

The front end of the hauling cord 30 is winded outside the cord rolling wheel 23 of the stretching mechanism 20, and the terminal end goes through the cord-guiding hole 212 of the separating board 21 and out of the enclosure 10. On the front end of the hauling cord 30, a clasping part 301 is formed.

The hanging unit 40 is configured on the connecting part 211 of the separating board 21 of the stretching mechanism 20.

The two-way pulling cord 50 is a flexible cord and can be pulled in both directions. The front end of the two-way pulling cord 50 goes into the first guiding hole 121 of the rear enclosure 12 and lays outside the driving wheel 25, and leads out from the second guiding hole 122 of the rear enclosure 12. When pulling the two-way pulling cord 50, the driving wheel 25 can be driven to rotate.

On one side of the peripheral of the cord rolling wheel 23, an inserting slot 231 is configured, and the clasping part 301 on the front end of the hauling cord 30 can be inserted into and fixed with the inserting slot 231.

On the peripheral of the driving wheel 25, multiple guiding slots 251 with even spacing are formed for the two-way pulling cord 50 to be engaged into.

The above structures constitute the design of the present invention. The operational situation of the present invention is described in detail below:

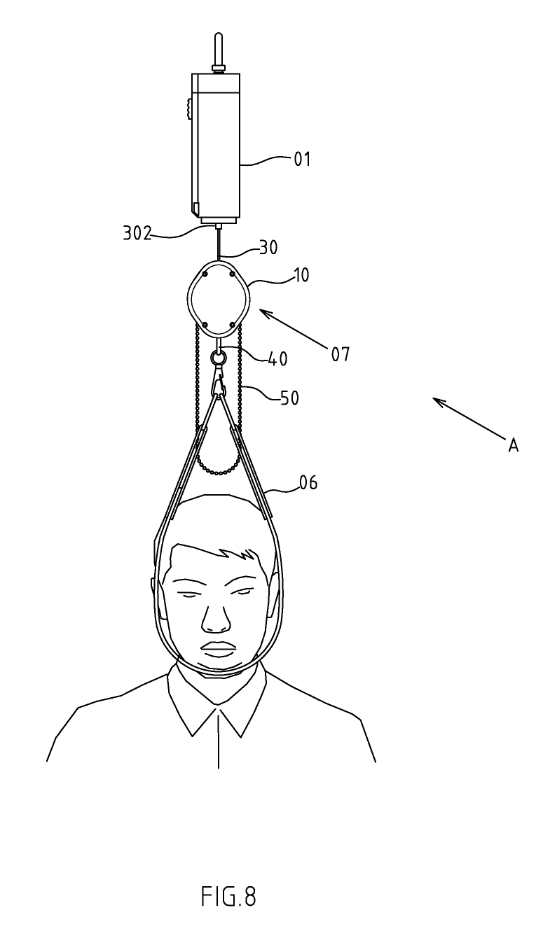

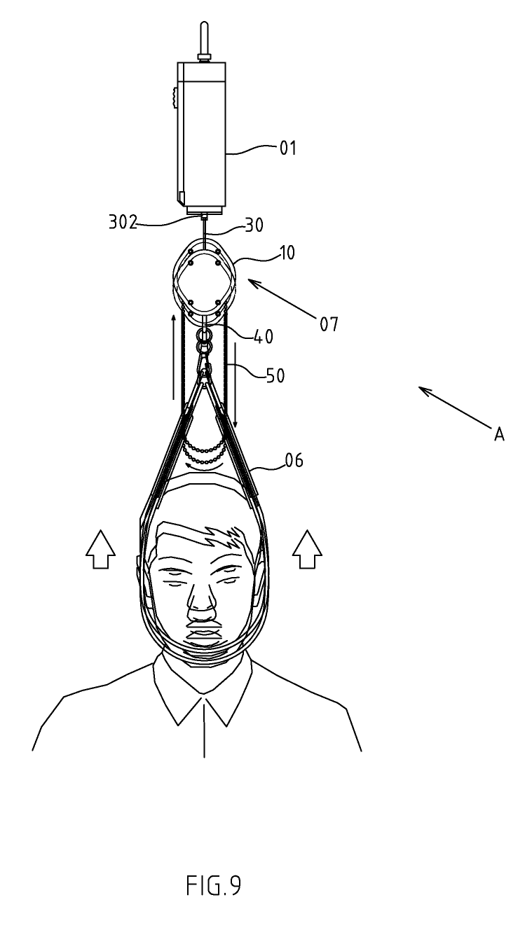

Referring to FIGS. 8 and 9, when using the body hauling mechanism A to improve the symptoms caused by irritation or compression of body nerves, the user connects the connecting part 302 at the terminal end of the hauling cord 30 to the lower end of the weight seat 01 already hanged and fixed. The hanging unit 40 at the lower end of the weight control assembly 07 hangs a cervical vertebrae hauling strap 06. The weight seat 01 can tell the hauling load on the body hauling auxiliary part 06 applied by the weight control assembly 07. When using the body hauling mechanism A, the body hauling auxiliary part 06 is looped onto the mentum of the user having the cervical vertebrae symptoms, and then the two-way pulling cord 50 is pulled by hand so that the weight control assembly 07 hauls and lifts the cervical vertebrae hauling strap 06 hanged on the hanging unit 40 at a mild speed, and generates a hauling force on the affected part of the user having cervical vertebrae symptoms. During the process of pulling the two-way pulling cord 50, because the ball chain-shaped two-way pulling cord 50 is embedded into the guiding slots 251 configured on the driving wheel 25, it will drive the driving wheel 25 to generate a rotating torsion, which is transmitted to the gear unit 22 through the driving axis 221, and increased through the gear ratio of the gear unit 22. The increased torsion further drives the cord rolling wheel 23 configured on the driving axis 221 to retract the hauling cord 30. As a result of the retraction of the hauling cord 30, the body hauling mechanism A generates an upward hauling force, and pulls the body hauling auxiliary part 06 configured on the hanging unit 40, and applies a preset hauling load on the affected part of the user having cervical vertebrae symptoms. Moreover, as the stretching mechanism 20 is configured with a back-stop mechanism 24, the body hauling mechanism A being hauled upward will not slip downward. To release the hauling force on the affected part of the user having cervical vertebrae symptoms, the two-way pulling cord 50 can be pulled in the opposite direction for lowering at a mild speed. In this way, the mechanism can help improve the symptoms caused by irritation or compression of body nerves without causing secondary injury in the neck when the release is too speedy.

1. A body hauling mechanism comprising:
a weight seat, formed outside with an outer seat, and inside fitted with an inner seat which can slip out of the bottom end of the outer seat, and, between the inner seat and outer seat, a spring weight mechanism is configured;
a weight control assembly, comprising:
an enclosure, comprising a front enclosure and a rear enclosure corresponding to each other and fixed together, and inside the front enclosure and rear enclosure, a housing space is formed, on one side of the rear enclosure, there is a first guiding hole and a second guiding hole;
a stretching mechanism, configured inside the housing space of the enclosure;
a hauling cord, configured on a preset position of the stretching mechanism, and formed on the end with a connecting part, the connecting part is connected and fixed to the bottom of the inner seat of the weight seat, through activation of the stretching mechanism, the hauling cord can be winded up or released;
a hanging unit, configured on a preset position of the stretching mechanism, for the weight control assembly to hang a body hauling auxiliary part;
a two-way pulling cord, configured on a preset position of the stretching mechanism, which is a flexible cord and can be pulled in both directions; by pulling the two-way pulling cord, the stretching mechanism can be driven to wind up or release the hauling cord, so that the weight control assembly can generate a hauling force forward or backward;
the user can pull the two-way pulling cord in either directions to drive the stretching mechanism to control the weight control assembly hanged on the weight seat, so that the hauling cord of the weight control assembly can be retracted or released at a mild speed to generate or release the hauling force.

2. The structure defined in claim 1, wherein the two-way pulling cord is structured in a chain of beads with uniform spacing, with its front end and terminal end connected together to form a loop.

3. The structure defined in claim 1, wherein the stretching mechanism of the weight control assembly comprises: a separating board, a gear unit, a cord rolling wheel, a back-stop mechanism and a driving wheel; the separating board is fitted on the part where the front and rear enclosure are assembled together; on one side of the enclosure, a connecting part is configured; on the side of the connecting part, a cord-guiding hole is configured; the gear unit is configured in front of the separating board, and on the rear end of the gear unit a driving axis is configured; inside the space formed by the separating board and the gear unit, a cord rolling wheel is configured; the back-stop mechanism is configured and fixed on the back side of the separating board and can rotate in one direction; on the back side of the back-stop mechanism, a driving wheel is configured and can rotate in both directions; the driving axis is serially connected to the cord rolling wheel, back-stop mechanism and driving wheel, so that the cord rolling wheel, back-stop mechanism and driving wheel can be driven to rotate simultaneously, and the stretching mechanism is entirely positioned inside the housing space of the enclosure;
the front end of the hauling cord is winded outside the cord rolling wheel of the stretching mechanism, and the terminal end goes through the cord-guiding hole of the separating board and out of the enclosure; on the front end of the hauling cord, a clasping part is formed;
the hanging unit is configured on the connecting part of the separating board of the stretching mechanism;
the two-way pulling cord is a flexible cord and can be pulled in both directions; the front end of the two-way pulling cord goes into the first guiding hole of the rear enclosure and lays outside the driving wheel, and leads out from the second guiding hole of the rear enclosure.

4. The structure defined in claim 2, wherein, on one side of the peripheral of the cord rolling wheel, an inserting slot is configured, and the clasping part on the front end of the hauling cord can be inserted into and fixed with the inserting slot.

5. The structure defined in claim 2, wherein, on the peripheral of the driving wheel, multiple guiding slots with even spacing are formed for the two-way pulling cord to be engaged into.
